(19) United States
(12) Patent Application Publication
(10) Pub. No.: US 2026/0359978 A1
(43) Pub. Date: Nov. 12, 2026

(54) SYSTEMS AND METHODS FOR AUTOMATING CONDITIONALLY INCREASED ACOUSTIC OUTPUT

(71) Applicant: GE Precision Healthcare LLC, Waukesha, WI (US)

(72) Inventors: Michael H. Wang, Brookfield, WI (US); Michael Washburn, Brookfield, WI (US); Scott Schoen, JR., Quincy, MA (US); Anthony E. Samir, Newton, MA (US); Abder-Raman Ali, Halifax (CA); Theodore T. Pierce, Newton, MA (US)

(73) Assignee: GE Precision Healthcare LLC, Waukesha, WI (US)

(21) Appl. No.: 19/202,962

(22) Filed: May 8, 2025

(51) Int. Cl. G06T 7/00; A61B 8/00; G06T 7/11

(52) U.S. Cl. CPC G06T 7/0012; A61B 8/485; G06T 7/11; G06T 2207/10132; G06T 2207/20081; G06T 2207/30056

(57) ABSTRACT

Systems and methods for automating conditionally increased acoustic output are described herein. In one example, a system includes a processing circuit including a processor coupled to a memory device storing instructions. The instructions, when executed, cause the processing circuit to perform operations including obtaining an ultrasound image depicting an organ; segmenting the ultrasound image, including extracting the organ depicted by the ultrasound image; determining a skin-to-capsule distance; identifying a measurement location; estimating a mechanical index of an acoustic output based on a comparison of the skin-to-capsule distance at the measurement location to a threshold distance; emitting the acoustic output at the measurement location according to the mechanical index; obtaining data in response to emitting the acoustic output; and repeating emission of the acoustic output according to the mechanical index based on determining that the mechanical index provides a sufficient acoustic output.

### FIELD

Embodiments of the subject matter disclosed herein relate to ultrasound imaging, and more particularly, to ultrasound shear wave elastography.

### BACKGROUND

Ultrasound shear wave elastography is a non-invasive technique for assessing organ stiffness, such as for a liver. More specifically, this technique measures shear wave speed in the organ, and thus relies on the generation and tracking of shear waves within the organ. In an example process, an acoustic push pulse is used to impart a force on the organ tissue, which induces shear waves that travel outward from the push location. The shear waves are then tracked using ultrasound imaging and a shear wave speed is computed by estimating the time delays between adjacent points.

### SUMMARY

An embodiment relates to a system. The system includes a processing circuit including a processor coupled to a memory device storing instructions thereon that, when executed, cause the processing circuit to perform operations. The operations include obtaining an ultrasound image depicting an organ of a patient. The operations include segmenting the ultrasound image, where segmenting the ultrasound image includes extracting the organ depicted by the ultrasound image. The operations include determining, using the segmented ultrasound image, a skin-to-capsule distance. The operations include identifying, using the segmented ultrasound image, a measurement location. The operations include estimating a mechanical index of an acoustic output based on a comparison of the skin-to-capsule distance at the measurement location to a threshold distance. The operations include emitting the acoustic output at the measurement location according to the mechanical index. The operations include obtaining data in response to emitting the acoustic output. The operations include repeating emission of the acoustic output according to the mechanical index based on determining that the mechanical index provides a sufficient acoustic output based on the data.

Another embodiment relates to a system. The system includes an ultrasound probe and a transducer. The transducer is configured to obtain ultrasound image data of a patient. The system includes a processing circuit including a processor coupled to a memory device storing instructions thereon that, when executed, cause the processing circuit to perform operations. The operations include obtaining an ultrasound image depicting a liver of the patient. The operations include receiving data regarding a body mass index of the patient. The operations include segmenting the ultrasound image, where segmenting the ultrasound image includes extracting the liver depicted by the ultrasound image. The operations include determining, using the segmented ultrasound image, a skin-to-capsule distance. The operations include identifying, using the segmented ultrasound image, a measurement location. The operations include estimating a mechanical index of an acoustic output based on at least one of a comparison of the skin-to-capsule distance at the measurement location to a threshold distance or a comparison of the body mass index of the patient to a threshold index. The operations include emitting the acoustic output at the measurement location according to the mechanical index. The operations include obtaining shear wave elastography data in response to emitting the acoustic output. The operations include repeating emission of the acoustic output according to the mechanical index based on determining that the mechanical index provides a sufficient force of the acoustic output based on the shear wave elastography data.

Another embodiment relates to a method. The method includes obtaining, by a processing circuit, an ultrasound image depicting a liver of a patient. The method includes segmenting, by the processing circuit, the ultrasound image, where segmenting the ultrasound image includes extracting the liver depicted by the ultrasound image. The method includes determining, by the processing circuit using the segmented ultrasound image, a skin-to-capsule distance. The method includes identifying, by the processing circuit using the segmented ultrasound image, a measurement location. The method includes estimating, by the processing circuit, a mechanical index of an acoustic output based on a comparison of the skin-to-capsule distance at the measurement location to a threshold distance. The method includes emitting, by the processing circuit, the acoustic output at the measurement location according to the mechanical index. The method includes obtaining, by the processing circuit, shear wave elastography data in response to emitting the acoustic output. The method includes repeating, by the processing circuit, emission of the acoustic output according to the mechanical index based on determining that the mechanical index provides a sufficient force of the acoustic output based on the shear wave elastography data.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE DRAWINGS

FIG. 1 is a block diagram of an ultrasound imaging system, according to an example embodiment.

FIG. 2 is a block diagram of a processing circuit of the ultrasound imaging system of FIG. 1, according to an example embodiment.

FIG. 3A is an ultrasound image depicting a liver, according to an example embodiment.

FIG. 3B is an illustration of a segmentation of the ultrasound image of FIG. 3A, according to an example embodiment.

FIG. 4 is a diagram illustrating shear wave elastography data resulting from use of a conventional mechanical index versus an increased mechanical index, according to an example embodiment.

FIG. 5 is a graphical representation of shear wave elastography success when using a conventional mechanical index versus an increased mechanical index, according to an example embodiment.

FIG. 6 is a flow chart illustrating a method for performing a shear wave elastography process, according to an example embodiment.

FIG. 7 is a flow chart illustrating a method for iteratively performing the shear wave elastography process of FIG. 6, according to an example embodiment.

FIG. 8 is a flow chart illustrating a method for determining a mechanical index based on a skin-to-capsule distance, according to an example embodiment.

FIG. 9 is a flow chart illustrating a method for determining a mechanical index based on a body mass index, according to an example embodiment.

### DETAILED DESCRIPTION

Referring generally to the figures, systems and methods for automating conditionally increased acoustic output during a shear wave elastography process are disclosed. More specifically, the systems and methods described herein include tailoring an acoustic push pulse's mechanical index to patient-specific geometry, while using a safely increased level to reduce non-diagnostic acquisitions. While many of the examples herein relate to assessing stiffness in a liver, it will be appreciated that the techniques disclosed herein could be used to assess stiffness, or other characteristics, in other organs as well.

In subjects with higher body mass indices (BMIs), the performance of shear wave elastography is often degraded. For example, due to the larger skin-to-capsule distances (SCDs) that are typical in subjects with higher BMIs, the acoustic energy is aberrated and attenuated by thicker layers of subcutaneous fat. In such instances, however, stronger acoustic pushes (e.g., those having a mechanical index above the current regulatory limit of 1.9) have demonstrated an ability to improve measurement quality and consistency in subjects with higher BMIs, with no apparent effect on liver function. For subjects with smaller SCDs (e.g., typical SCDs), the measurements are reliable with smaller mechanical indices, and thus the stronger acoustic output is not needed. Even in these subjects with smaller SCDs, however, the stronger acoustic output has no apparent effect on liver function.

In existing systems used to perform shear wave elastography, adjusting the mechanical index, e.g., to use stronger acoustic pushes used during a shear wave elastography process on subjects with higher BMIs, relies on operator discretion and manual selection of appropriate settings.

The systems and methods described herein, however, provide a technical solution to existing systems by providing an automated process for identifying subjects that may benefit from the use of an increased acoustic output and determining the appropriate mechanical index of such increased acoustic outputs. M ore specifically, as described herein, deep learning is used to segment the liver capsule using a B-mode ultrasound image, enabling automatic measurements of the SCD, as well as automatically providing the geometry of the intervening subcutaneous fat layer. As such, the systems and methods described herein provide a fully automated conditionally-increased acoustic output technology configured to improve measurement quality safely, reliably, and consistently.

The implementations described herein address a technical problem by providing enhanced data integration and analysis capabilities, which deliver a particular technical solution that streamlines and refines performance of shear wave elastography. More specifically, the systems and methods described herein provide automatically determining a mechanical index used during shear wave elastography based on patient-specific parameters such as SCD, BMI, or tissue morphology. Furthermore, such systems and methods are configured to automatically conditionally increase the mechanical index until successful shear wave elastography measurements are obtained. Accordingly, this approach provides a specific technical improvement to various technical problems, including those set forth herein.

The systems described herein may also reduce processing power by performing various processing operations simultaneously, rather than performing a plurality of processing operations individually and consuming unnecessary processing power. That is, the systems and methods described herein result in more efficient performance of shear wave elastography procedures. For example, with the automated conditionally-increased acoustic output technology described herein, the ultrasound imaging system determines a mechanical index using an automatically segmented ultrasound image (e.g., segmented using deep learning, as described herein). Then, the system is configured to automatically increase the mechanical index incrementally until successful measurements are obtained, rather than relying on manually entered mechanical indices and sonographer expertise in estimating an extent with which to increase the mechanical index.

The disclosures of U.S. patent application Ser. No. 18/205,809, published as U.S. Publication No. 2024/0398382, and entitled Ultrasound Imaging Techniques For Shear-Wave Elastography, and U.S. patent application Ser. No. 18/205,804, published as U.S. Publication No. 2024/0404263, and entitled Ultrasound Image Feature Segmentation, are hereby incorporated by reference in their entirety.

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

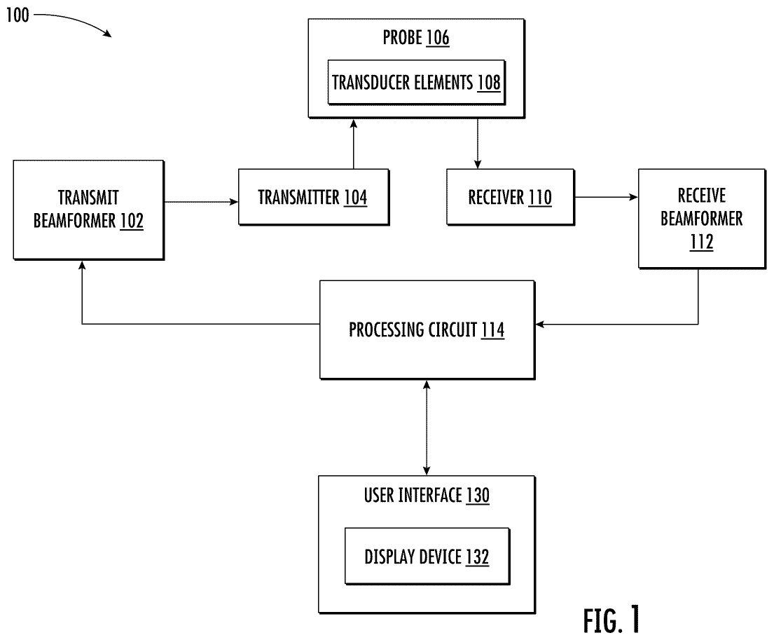

Referring to FIG. 1, a block diagram of an ultrasound imaging system 100 is shown. The ultrasound imaging system 100 may be used in a medical environment (e.g., hospitals, clinics, etc.), for example, by a sonographer, technician, or other clinician certified to collect ultrasound data from a patient. In some embodiments, the ultrasound imaging system 100 may be operable to perform a shear-wave elastography process, for example, to obtain information about liver tissue. It should be appreciated that in some instances, conventional ultrasound imaging and shear-wave elastography may be performed by a single ultrasound imaging system 100, while in other instances, the conventional ultrasound imaging and the shear-wave elastography may be performed by a plurality of ultrasound imaging systems 100.

An example of a procedure performed using the ultrasound imaging system 100 may be a shear wave elastography procedure. As a brief overview, a shear wave elastography procedure may begin with a sonographer selecting a suitable imaging modality of the ultrasound imaging system 100 (e.g., a liver setting). Once a patient is in position for the procedure (e.g., lying on their back with the right arm raised), the sonographer proceeds to collect ultrasound data of the liver using the ultrasound imaging system 100 according to the suitable imaging modality. The sonographer identifies an anatomical region or feature (e.g., the liver) using the ultrasound data. Once the region or feature is identified, the sonographer activates an elastography mode of the ultrasound imaging system 100 such that the ultrasound imaging system 100 emits high-intensity pulses configured to generate shear waves within the tissue. Collection of the shear wave elastography data is repeated to ensure precise measurements.

As shown in FIG. 1, the ultrasound imaging system 100 includes a transmit beamformer 102, a transmitter 104, a probe 106, a receiver 110, and a receive beamformer 112.

The transmit beamformer 102 may be either a hardware beamformer or a software beamformer. In embodiments where the transmit beamformer 102 is a hardware beamformer, the transmit beamformer 102 may include one or more of a graphics processing unit (GPU), a microprocessor, a central processing unit (CPU), a digital signal processor (DSP), or any other type of processor capable of performing logical operations. The transmit beamformer 102 may be configured to perform conventional beamforming techniques as well as techniques such as retrospective transmit beamforming (RTB). Alternatively, in embodiments where the transmit beamformer 102 is a software beamformer, a processor (e.g., processor 116, as described below) may be configured to perform some or all of the functions associated with the transmit beamformer 102.

The probe 106 may be a linear array probe, a curvilinear array probe, a sector probe, or any other type of probe configured to obtain two-dimensional (2D) B-mode data, 2D color flow data, M-mode data, three-dimensional (3D) data, four-dimensional (4D) data, or any other type of ultrasound data. Alternatively or additionally, the probe 106 may be any type of probe configured to obtain 2D B-mode data and data corresponding to another ultrasound mode that detects blood flow velocity in the direction of a vessel axis. In some embodiments, the probe 106 may include a position sensor configured to detect a position of the probe 106 relative to one or more reference locations. That is, the position sensor may continuously track movement (e.g., rotation, translation, orientation, etc.) of the probe 106 relative to the location of the probe 106 when the anatomy being imaged is identified. For example, the anatomy being imaged may be identified as a liver at a first location of the probe 106. Then, the position sensor may track the movement of the probe 106 relative to the liver in order to identify successive locations of the probe 106. In some embodiments, the position sensor may transmit position data to be stored within the ultrasound imaging system 100 (e.g., in memory 118).

The probe 106 may include a transducer configured to transmit and receive an ultrasound signal. In some embodiments, as shown in FIG. 1, the probe 106 includes transducer elements 108. The transducer elements 108 may be arranged in a transducer array, and in some embodiments may be arranged in a one-dimensional (1D) or 2D array. The transmit beamformer 102 and the transmitter 104 drive the transducer elements 108 to emit pulsed ultrasonic signals into a body of a subject (e.g., a patient). For example, during a liver examination, a sonographer or other clinician may navigate the probe 106 proximate to a patient's liver so that the transducer elements 108 in the probe 106 emit the pulsed ultrasonic signals into the patient's liver. The pulsed ultrasonic signals are then back-scattered from anatomical structures in the body, such as blood cells or muscular tissues, to produce echoes that return to the transducer elements 108. That is, the transducer elements 108 may include the transducer configured to transmit and receive the ultrasound signal, a matching layer configured to have an acoustic impedance between a tissue to be imaged and a material of the transducer (e.g., such that the pulsed electronic signals can be back-scattered from the anatomical structures in the body and received as echoes by the transducer elements 108), and a damping block configured to absorb ultrasound energy.

In some embodiments, the probe 106 may be used to perform a shear wave elastography procedure, as described herein. That is, the probe 106 may be used to provide an external force (e.g., via a transducer, a vibration device, etc.) that creates shear waves in a patient's tissue (e.g., liver tissue, tissues or structures proximate to the liver tissue, etc.). In some instances, the transducer configured to provide the external force may be external to the ultrasound imaging system 100. Additionally or alternatively, the ultrasound imaging system 100 may provide a push force such as a relatively high-intensity ultrasonic push pulse for generating the shear waves in the tissue.

As an example, the transmitter 104 may be configured to (e.g., through a sub-aperture transmission) drive the transducer elements 108 to emit relatively high-intensity ultrasound push pulses into a point of disturbance of the tissue, and to emit ultrasonic transmit signals into the tissue. As used herein, the term “high-intensity ultrasound push pulses” refers to a derated spatial-peak temporal-average intensity (ISPTA.3) of between 200 and 700 mW/cm². The transmitted high-intensity ultrasound push pulses may displace the tissue to create shear waves that propagate laterally from the point of disturbance. The transmitted ultrasonic signals may be reflected from structures, such as the tissue deformed by the shear waves, to produce echoes. As described below, the echoes are received by the receiver 110. The receiver 110 may be configured to convert the received echoes into analog signals, which undergo beamforming by the receive beamformer 112.

The receiver 110 receives the echoes from the probe 106 and converts the echoes into electrical signals. The electrical signals are then passed through the receive beamformer 112, which produces the ultrasound data from the electrical signals. As described above with reference to the transmit beamformer 102, the receive beamformer 112 may be either a hardware beamformer or a software beamformer. In embodiments where the receive beamformer 112 is a hardware beamformer, the receive beamformer 112 may include one or more of a GPU, a microprocessor, a CPU, a DSP, or any other type of processor capable of performing logical operations. The receive beamformer 112 may be configured to perform conventional beamforming techniques as well as techniques such as retrospective transmit beamforming (RTB). Alternatively, in embodiments where the receive beamformer 112 is a software beamformer, a processor (e.g., processor 116, as described below) may be configured to perform some or all of the functions associated with the receive beamformer 112.

Although the transmit beamformer 102, the transmitter 104, the receiver 110, and the receive beamformer 112 are shown in FIG. 1 as being components of the ultrasound imaging system 100 that are distinct from the probe 106, it should be appreciated that in some embodiments, the probe 106 may include electronic circuitry configured to perform the functions of each of the transmit beamformer 102, the transmitter 104, the receiver 110, and/or the receive beamformer 112. That is, all or part of the transmit beamformer 102, the transmitter 104, the receiver 110, and/or the receive beamformer 112 may be situated within the probe 106.

Referring still to FIG. 1, the ultrasound imaging system 100 is shown to include a processing circuit 114. While shown as being separate from the probe 106 in FIG. 1, it will be appreciated that the processing circuit 114 can be part of the probe 106. For example, the processing circuit 114 can be disposed in a handheld housing of the probe 106 (e.g., in the case of the probe 106 being a wireless probe).

The ultrasound imaging system 100 is also shown to include a user interface 130. The user interface 130 may be used by a sonographer or other clinician to control operation of the ultrasound imaging system 100. For example, the sonographer may use the user interface 130 to initiate shear-wave elastography imaging, control the input of patient data, to change a scanning or display parameter, to adjust a segmentation of an anatomical feature depicted in an ultrasound image, and/or to select various other modes, operations, parameters, etc. of the ultrasound imaging system 100. In some embodiments, the user interface 130 may include an off-the-shelf consumer electronic device such as a smartphone, a tablet, a laptop, and so on. For the purposes of this disclosure, the term “off-the-shelf consumer electronic device” is defined to be an electronic device that was designed and developed for general consumer use and one that was not specifically designed for use in a medical environment. Alternatively, in other embodiments, the user interface 130 may be an electronic device that was designed and developed for use in a medical environment.

According to some embodiments, the user interface 130 may be physically separate from the rest of the ultrasound imaging system 100 (e.g., the transmit beamformer 102, the transmitter 104, the probe 106, the receiver 110, the receive beamformer 112, or the processing circuit 114). The user interface 130 may communicate with the processing circuit 114 (e.g., processor 116) through a wireless protocol, such as Wi-Fi, Bluetooth, wireless local area network (WLAN), near-field communication, and so on. According to some embodiments, the user interface 130 may communicate with the processing circuit 114 through an application programming interface (API).

In some embodiments, the user interface 130 may include physical controls such as one or more of buttons, sliders, a rotary knob, a mouse, a keyboard, a trackball, hard keys linked to specific actions, soft keys that may be configured to control different functions, and so on. As shown in FIG. 1, the user interface 130 may also include a display device 132. In some embodiments, the display device 132 may be configured to display a graphical user interface (GUI) based on an instruction from a system memory (e.g., memory 118). The GUI may include user interface icons representing commands and instructions relating to the operation of the ultrasound imaging system 100. The user interface icons of the GUI may be configured such that a user (e.g., the sonographer, clinician, etc.) may select a specific user interface icon in order to initiate a specific function controlled by the GUI. For example, various user interface icons may be used to represent windows, menus, buttons, cursors, scroll bars, and so on. That is, the physical controls of the user interface 130 may be included as individual hardware elements, as user interface icons displayed on the display device 132, or as a combination of hardware elements and user interface icons.

In some embodiments, the display device 132 may include a touch-sensitive display device or a touch screen. According to such embodiments, the touch screen may be configured to interact with the GUI displayed by the display device 132 such that a user (e.g., the sonographer) can interact with the GUI via the touch screen. The touch screen may be a single-point touch screen that is configured to detect a single contact point at a time, or the touch screen may be a multi-point touch screen that is configured to detect multiple points of contact at a time. For embodiments where the touch screen is a multi-point touch screen, the touch screen may be configured to detect multi-point gestures involving contact from two or more of a user's fingers at a time. The touch screen may be a resistive touch screen, a capacitive touch screen, or any other type of touch screen that is configured to receive inputs from a stylus or one or more of a user's fingers. According to some embodiments, the touch screen may be an optical touch screen that uses technology such as infrared light or other frequencies of light to detect one or more points of contact initiated by a user. In some embodiments, the touch screen may be incorporated as part of the display device 132 or may be separate from the display device 132. The user interface 130 may also include a proximity sensor configured to detect objects and/or gestures that are within a predetermined distance (e.g., five feet, six inches, ten centimeters, etc.) of the proximity sensor. In various embodiments, the proximity sensor may be located on the display device 132 or as part of a touch screen that is separate from the display device 132.

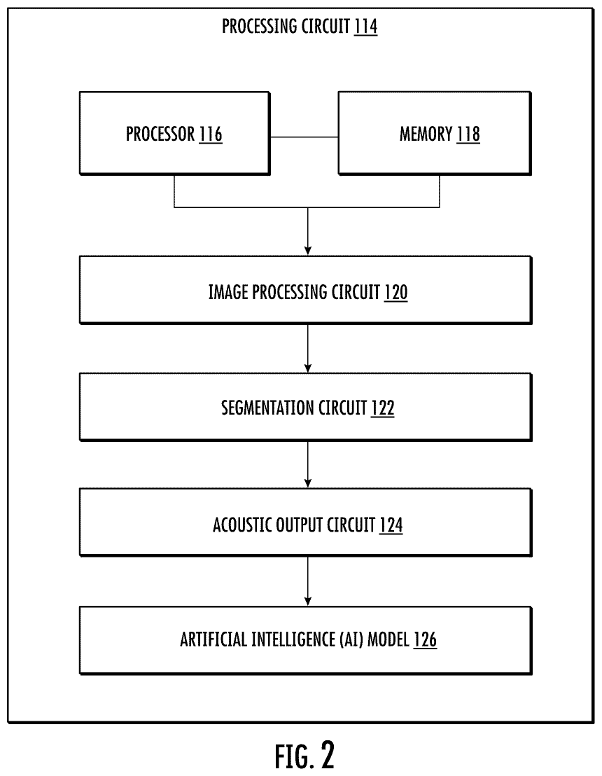

Referring to FIG. 2, the processing circuit 114 of the ultrasound imaging system 100 is shown in greater detail. As shown, the processing circuit 114 may include at least one processor 116, a memory 118, an image processing circuit 120, a segmentation circuit 122, an acoustic output circuit 124, and an artificial intelligence (AI) model 126. In this way, the processing circuit 114 may be structured or configured to execute or implement the instructions, commands, and/or control processes described herein with respect to the processor 116, the memory 118, the image processing circuit 120, the segmentation circuit 122, the acoustic output circuit 124, and the AI model 126.

The processor 116 may include a CPU, a GPU, a microprocessor, a DSP, a general-purpose single-or multi-chip processor, a field-programmable gate array (FPGA), or any other type of processor capable of performing logical operations. A general-purpose processor may be a microprocessor, or, any conventional processor, or state machine. A processor also may be implemented as a combination of computing devices, such as a combination of a DSP and a microprocessor, a plurality of microprocessors, one or more microprocessors in conjunction with a DSP core, or any other such configuration. In some embodiments, the processor 116 may be shared by multiple circuits (e.g., the circuits of the processor 116 may include or otherwise share the same processor which, in some example embodiments, may execute instructions stored, or otherwise accessed, via different areas of the memory 118). Alternatively or additionally, the processor 116 may be structured to perform or otherwise execute certain operations independent of one or more co-processors. In some embodiments, two or more processors may be coupled via a bus to enable independent, parallel, pipelined, or multi-threaded instruction execution. All such variations are intended to fall within the scope of the present disclosure.

The processor 116 may be configured to control the transmit beamformer 102, the transmitter 104, the receiver 110, and the receive beamformer 112. The processor 116 may also be in electronic communication with the probe 106. For purposes of this disclosure, the term “electronic communication” may be defined to include both wired and wireless communications.

In some embodiments, the processor 116 may be configured to control the probe 106 during data acquisition. That is, the processor 116 may control the data acquisition by controlling which of the transducer elements 108 are active and by controlling a shape of the beam emitted from the probe 106. Alternatively or additionally, the processor 116 may include a complex demodulator configured to demodulate radio frequency (RF) data obtained by the probe 106 and generate raw data. According to other embodiments, the demodulation of the RF data may be performed by another component of the ultrasound imaging system 100. The processor 116 may perform the processing operations described herein according to a plurality of selectable ultrasound modalities.

Depending on a mode of operation of the ultrasound imaging system 100, the processor 116 may process ultrasound data obtained by the probe 106 according to the mode of operation to generate 2D or 3D image data. For example, the mode of operation may include B-mode, color flow Doppler mode, M-mode, color M-mode, spectral Doppler, elastography, TVI, strain, strain rate, and the like. Various of these modes of operation may be configured to, for instance, convert ultrasound data from beam space coordinates (e.g., received from the receive beamformer 112) to display space coordinates (e.g., such that the ultrasound data may be displayed as image data). In some embodiments, the mode of operation may allow for video processing by the processor 116 such that a series of images (e.g., processed ultrasound data) may be displayed in real-time while a scanning session/procedure is being performed on a patient. An operator of the ultrasound imaging system 100 (e.g., a sonographer) may switch between various modes in order to obtain a variety of ultrasound data and to perform a complete scan of an anatomical region. For example, the operator may switch between modes using the user interface 130 (e.g., using physical controls, interface inputs representing physical controls, etc.). While the term “image” or “images” are used herein to for the purposes of example, it will be appreciated that such terms cover still images as well as videos, clips, or a series of images for each. For example, in some embodiments, the image or images may include a 1-2 second clip derived from the image data.

The processor 116 performs the processing operations in real-time as the echo signals are received by the receiver 110 from the probe 106. For the purposes of this disclosure, the term “real-time” is defined to include a procedure that is performed without any intentional delay. As an illustrative, non-limiting example, in certain instances, the ultrasound imaging system 100 may obtain images at a real-time volume-rate of 7-20 volumes/sec. It should be appreciated, however, that the real-time volume-rate may be dependent on the length of time that it takes to obtain each volume of data for display. Thus, the ultrasound imaging system 100 may be configured to obtain 2D data of an anatomical region at a faster rate than 3D data of the same anatomical region because it takes longer to obtain a volume of 3D data than the same volume of 2D data. Similarly, when the ultrasound imaging system 100 obtains a relatively large volume of data, the real-time volume-rate may be slower than for a smaller volume of data. For example, during an abdominal scan, the real-time volume-rate may be slower if the patient is an adult versus if the patient is an infant because the volume of data is larger for the adult than for the infant (e.g., due to the abdomen of an adult being larger than the abdomen of an infant). Therefore, certain implementations of the ultrasound imaging system 100 may have real-time volume-rates that are faster than 20 volumes/sec, while other implementations of the ultrasound imaging system 100 may have real-time volume-rates that are slower than 7 volumes/sec.

In some embodiments, the processor 116 may be configured to process shear wave ultrasound data to provide information about an anatomical region or feature, such as a liver. As used herein, the term “shear-wave ultrasound data” refers to ultrasound information received by the processing circuit 114 corresponding to the echoes produced by the back-scattering of the transmitted ultrasonic signals from structures (e.g., tissue) and surgical instruments in the object of interest as deformed by the shear waves. That is, the processor 116 may be configured to receive shear-wave elastography measurements during a shear wave elastography process (e.g., measurements convertible into image data or to determine other characteristics of human anatomy, such as liver stiffness related to metabolic dysfunction-associated steatotic liver disease).

In this regard, the processor 116 may be configured to facilitate processing the shear wave ultrasound data to determine a local distribution of shear wave speed in the tissue. The shear-wave speed may be computed by direct inversion of the Helmholtz equation, time-of-flight measurement, or any suitable computational method. The shear-wave ultrasound data may be acquired after a push disturbance is induced in the tissue by the force of a focused ultrasound beam or by an external push force, for example. The push disturbance generates shear waves that propagate laterally from the point of disturbance. The ultrasound imaging system 100 acquires the shear-wave ultrasound data using a high pulse repetition frequency. As used herein, the term “high-pulse repetition frequency” refers to a pulse repletion frequency of at least 300 pulses/second. In some embodiments, the pulse repetition frequency used to acquire shear-wave ultrasound data is greater or equal to 1000 pulses/second. Therefore, it should be appreciated that shear-wave elastography imaging may have higher frame rates than conventional ultrasound imaging due to the high pulse-repetition frequency used to image shear waves in a patient's tissue.

Furthermore, the processor 116 may be configured to convert the local distribution of shear wave speed in the tissue to a map, such as a velocity distribution map, an elasticity map, a spatial gradient map, or any suitable map representing the contrast between a lesion and surrounding tissue. For example, the local distribution may be mapped based on the shear-wave speed to generate a velocity distribution map. As another example, the local distribution may be converted to the elasticity map by computing the stiffness based on Young's modulus, a similar shear modulus, or any suitable conversion computation. Moreover, in various embodiments, a spatial gradient filter may be applied to the velocity distribution map and/or elasticity map to generate a spatial gradient map providing enhanced visualization tissue of interest.

The map represents the speed that the shear wave passed through the tissue at lateral locations from the point of disturbance in the shear-wave ultrasound data. The shear-wave propagation velocity corresponds to the stiffness of the tissue at the lateral locations. Specifically, the higher shear-wave velocity corresponds with more stiffness and the lower shear-wave velocity corresponds with less stiffness. Based on the difference in velocity and/or elasticity of tissue, the stiffness of the liver can be assessed. For example, the maps may be color-coded or grayscale maps having a range of colors or grays that correspond with the shear-wave speed and/or elasticity. Specifically, an elasticity map may have dark blue or dark gray/black corresponding with soft elasticity to red or light gray/white corresponding with hard elasticity, among other things. The map having the elasticity information may be overlaid on an ultrasound image such as a B-mode image or any suitable ultrasound image.

In some embodiments, the ultrasound imaging system 100 may include multiple processors configured to perform the processing operations/functionality described with reference to processor 116. For example, in such embodiments, a first processor of the multiple processors may be configured to demodulate and decimate the RF signal while a second processor of the multiple processors may be configured to further process the RF data prior to displaying an image representative of the data. It should be appreciated that other embodiments may use a different arrangement of processors.

The processor 116 may also be in electronic communication with the display device 132 such that the processor 116 may process ultrasound data obtained by the probe 106 and generate images to display on the display device 132.

As shown in FIG. 2, the processing circuit 114 also includes memory 118. The memory 118 may be configured to, for example, store processed volumes of data obtained by the ultrasound imaging system 100 (e.g., ultrasound data collected by the probe 106, user inputs received by the user interface 130, etc.). For example, the memory 118 may be a hospital picture archiving and communication system (PACS). The memory 118 (e.g., memory, memory unit, storage device, etc.) may include one or more devices (e.g., RAM, ROM, Flash memory, hard disk storage, etc.) for storing data and/or computer code for completing or facilitating the processes, layers, and modules described in the present application. The memory 118 may be or include tangible, non-transient volatile memory or non-volatile memory. The memory 118 may also include database components, object code components, script components, or any other type of information structure for supporting the activities and information structures described in the present application.

In various embodiments, the memory 118 may have varying capacity (e.g., storage space) across embodiments of the ultrasound imaging system 100. For example, the memory 118 may be configured to store at least 60 minutes' worth of ultrasound data. The ultrasound data may be stored in the memory 118 such that the ultrasound data may be retrieved according to an order/time of acquiring the data. That is, the ultrasound data may be stored with a timestamp indicating a time at which the ultrasound data was collected and may be retrieved starting with an oldest time at which the ultrasound data was collected.

The processing circuit 114 also includes the image processing circuit 120. The image processing circuit 120 is configured to receive image data obtained by the transducer of the probe 106 during an ultrasound scan. The image data refers to ultrasound data collected by the probe 106 while performing an ultrasound examination on a patient. For example, the image data may be collected during an ultrasound of a liver and may therefore include various images of a patient's liver and surrounding area. The image processing circuit 120 may include multiple deep learning-based models configured to analyze the image data. For example, the image processing circuit 120 may be configured to identify a view from which the image data is captured, an anatomical structure or other feature captured by the image data, the presence of a pathology in the image data, and so on. In some instances, the image processing circuit 120 may be configured to determine whether a desired view (e.g., a view of the liver) has been obtained based on the image data. The image processing circuit 120 may be configured to identify the anatomical structure using one or more algorithms (e.g., image processing algorithms such as edge detection, machine learning models, deep neural networks, etc.). In some embodiments, the image processing circuit 120 may identify anatomical features such as bones, blood vessels, organs, etc., based on a shape, relative proximity, apparent depth, orientation, etc. of said features in the image data.

The segmentation circuit 122 may be configured to segment ultrasound images, as described herein. In some instances, segmenting ultrasound images may include extracting an anatomical region or feature (e.g., an organ, etc.) from the ultrasound image. For example, the segmentation circuit 122 may be configured to extract a liver capsule or liver in ultrasound image data. As described in greater detail herein, the extracted anatomical region may indicate a location from where shear-wave elastography data is obtained. The shear-wave elastography measurements may indicate a stiffness of the patient's liver, and may be useful for diagnosing liver fibrosis.

In this regard, the segmentation circuit 122 may include, for example, artificial intelligence image analysis algorithms, one or more deep neural networks (e.g., a convolutional neural network such as ENet) and/or may utilize any suitable form of artificial intelligence image analysis techniques or machine learning processing functionality configured to provide automated segmentation functionality.

For example, the segmentation circuit 122 may include a deep neural network that may be made up of, for instance, an input layer, an output layer, and one or more hidden layers in between the input and output layers. Each of the layers may be made up of a plurality of processing nodes that may be referred to as neurons. For example, the input layer may include neurons corresponding to pixels of the obtained ultrasound image. The output layer may include neurons corresponding spatially to pixels in the obtained ultrasound image, where each neuron outputs a predicted anatomical classification at the respective pixel location indicating anatomical structures such as the liver, renal cortex, or any other suitable anatomical structure. Each neuron of each layer may perform a processing function and pass the processed ultrasound image information to one of a plurality of neurons of a downstream layer for further processing. As an example, neurons of a first layer may learn to recognize edges of a structure in an ultrasound image. The neurons of a second layer may learn to recognize shapes based on the detected edges from the first layer. The neurons of a third layer may learn positions of the recognized shapes relative to landmarks in the ultrasound image. As such, the processing performed by the deep neural network may identify anatomical structures and the location of the structures in the ultrasound image with a high degree of probability.

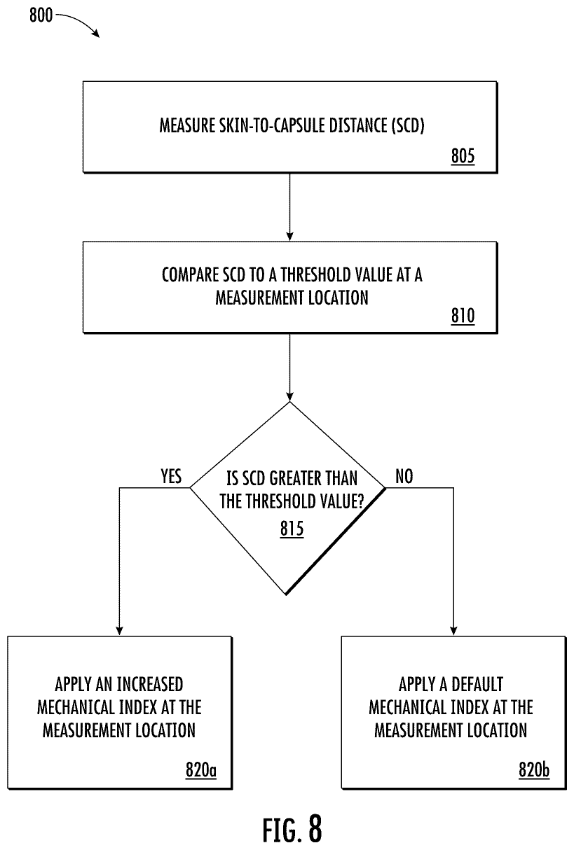

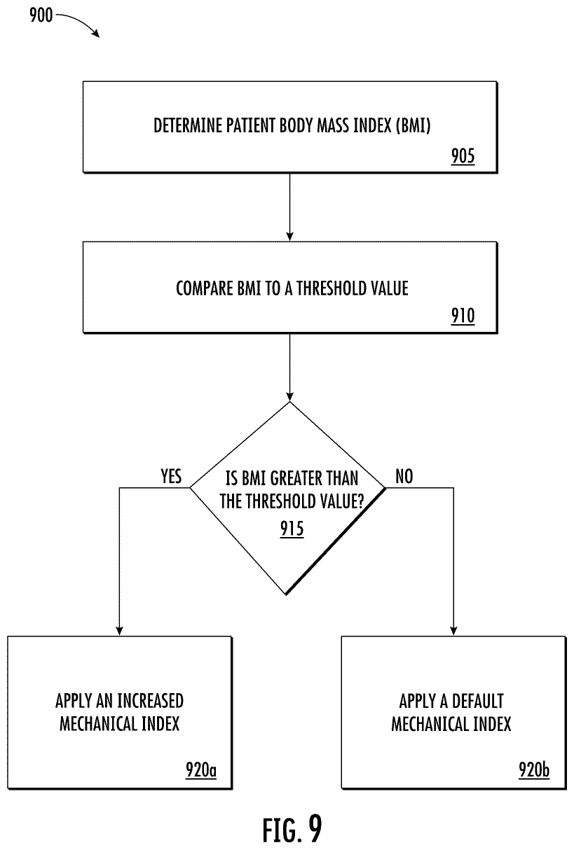

The acoustic output circuit 124 may be configured to cause the transducer (e.g., via the probe 106) to emit an acoustic output (e.g., push force, push pulse, etc.) according to a mechanical index. For example, the acoustic output may be emitted during a process to gather shear-wave elastography measurements from a patient's liver to measure the stiffness of the liver. Furthermore, the acoustic output circuit 124 may be configured to determine a mechanical index with which to emit the acoustic output, as described below. In some embodiments, the acoustic output circuit 124 may determine the mechanical index based on a patient's SCD, as described below with reference to FIG. 8. Additionally or alternatively, the acoustic output circuit 124 may determine the mechanical index based on a patient's BMI, as described below with reference to FIG. 9.

The AI model 126 refers to one or more AI models trained to perform the various operations relating to shear wave elastography described herein. The AI model 126 may be structured using a deep learning architecture that includes a multitude of interconnected layers. The AI model 126 may be trained on large datasets to assimilate patterns, structures, and relationships within the data. The trained AI model 126 can be trained to generate outputs that resemble or closely resemble the characteristics of the input data. The AI model 126 may be fine-tuned to generate specific output data, including data that is compatible with various database architectures or provider computing systems. The AI model 126 can be trained via optimization of a large number of parameters, in which the AI models learn to minimize the error between its predictions and the actual data points, resulting in highly accurate and coherent generative capabilities.

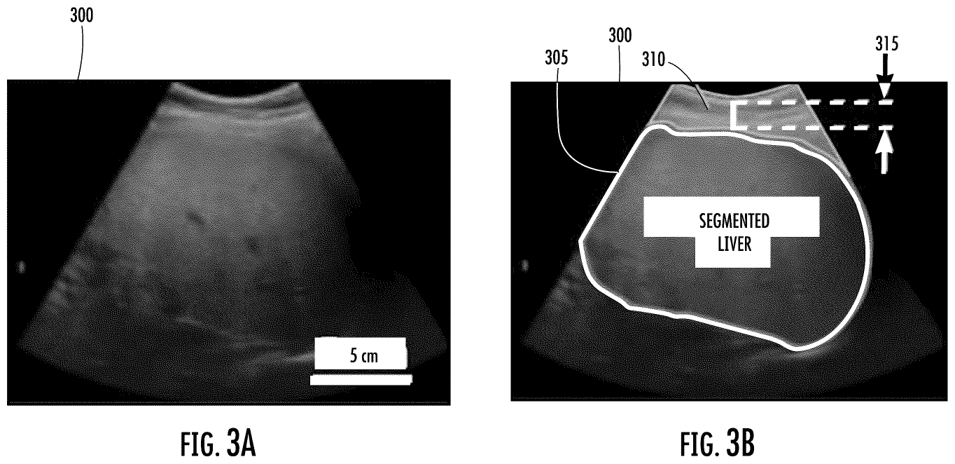

Referring to FIG. 3A, an ultrasound image 300 is shown. More specifically, the ultrasound image 300 depicts a liver and its surrounding structures (e.g., skin, tissues, etc.). In some embodiments, the ultrasound image 300 may be obtained by the ultrasound imaging system 100 (e.g., the probe 106) during an ultrasound imaging procedure. In some instances, the ultrasound image 300 may be obtained prior to performing a shear wave elastography process. In such instances, the ultrasound image 300 may be used to determine an anatomical region into which the shear waves are emitted. The ultrasound image 300 may be processed by the image processing circuit 120 to determine whether the ultrasound image 300 captures the desired view for the shear wave elastography procedure (e.g., depicting the liver, minimized presence of blood vessels, etc.).

Upon determining that the ultrasound image 300 captures the desired view of the liver, the segmentation circuit 122 may be configured to segment the ultrasound image 300. More specifically, as shown in FIG. 3B, the segmentation circuit 122 may segment a liver 305 and a fat layer 310 depicted by the ultrasound image 300. In some instances, the segmentation circuit 122 may determine the segmented liver 305 and the fat layer 310 substantially in real-time relative to when the ultrasound image 300 is generated. For example, the segmented liver 305 and the fat layer 310 may be determined within between 0.5 and 2 seconds (without limitation) after ultrasound image 300 is obtained. As described above, the segments (e.g., liver 305, fat layer 310) can be used to determine a location in the liver from which to track the propagation of shear waves as part of a shear-wave elastography process. For example, the segmented liver 305 can be used to automatically determine a location or possible location from which to process data (e.g., data generated by a shear-wave elastography process).

Furthermore, based on the segmented liver 305 and the segmented fat layer 310, the segmentation circuit 122 may be configured to determine an SCD 315. As shown in FIG. 3B, the SCD 315 refers to the minimum distance between the patient's epidermis and the liver capsule or liver. The upper extent of the liver or liver capsule may be determined by segmentation, as discussed above. The SCD 315 may be a measurement in length, for example, in millimeters. In some instances, the length of the SCD 315 may correlate with a patient's condition or measurement, such as body-mass index (BMI). It should be appreciated that a “skin-to-capsule” distance (e.g., SCD) refers to an estimation of a distance between the skin surface and the boundary of an anatomical structure. That is, some anatomical structures of interest (a pancreas, an ovary, a breast lesion, etc.) may not have a “capsule” in the biological sense. Such structures do, however, have a boundary that can be detected, and thus the distance from the skin to the boundary can be estimated. In this way, this distance from the skin to the boundary may be referred to as a “skin-to-capsule” distance, even though the structure may not have a biological “capsule.”

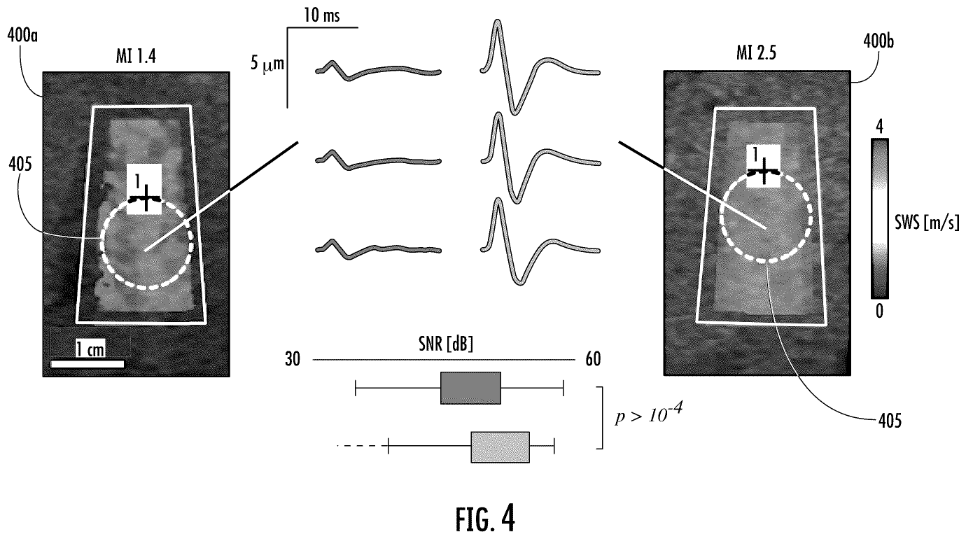

Referring to FIG. 4, a diagram is shown illustrating first shear wave elastography data 400a and second shear wave elastography data 400b. The first shear wave elastography data 400a depicts shear wave elastography data resulting from use of a relatively lower mechanical index (e.g., 1.4), while the second shear wave elastography data 400b depicts shear wave elastography data resulting from use of a relatively higher mechanical index (e.g., 2.5). The first shear wave elastography data 400a and the second shear wave elastography data 400b include a measured displacement 405. The measured displacement 405 refers to the displacement of the shear waves (e.g., in the liver tissue). As shown, the relatively higher mechanical index used to obtain the second shear wave elastography data 400b resulted in a higher signal-to-noise ratio (SNR) than the relatively lower mechanical index.

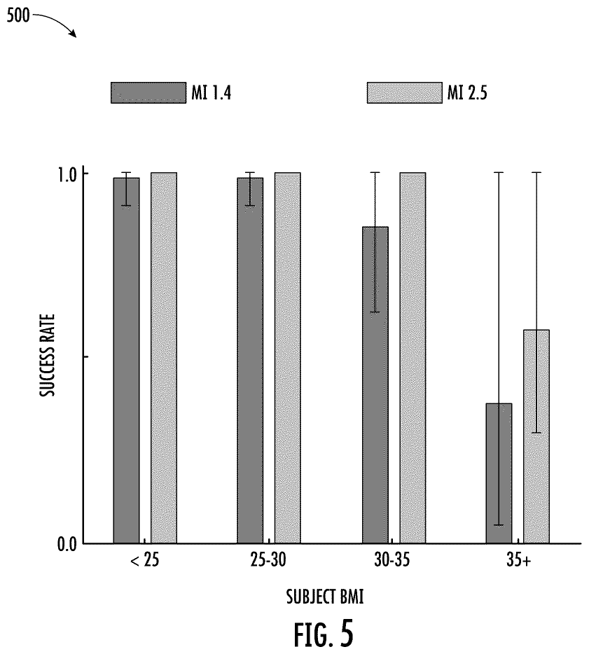

FIG. 5 depicts a graphical representation 500 of shear wave elastography success when using a relatively lower mechanical index versus a relatively higher mechanical index. As shown, the relatively lower mechanical index may be the mechanical index used to obtain the first shear wave elastography data 400a (e.g., 1.4), while the relatively higher mechanical index may be the mechanical index used to obtain the second shear wave elastography data 400b (e.g., 2.5). As shown by FIGS. 4-5, using the relatively higher mechanical index (e.g., 2.5) improves shear wave elastography measurement. For instance, FIG. 5 illustrates that for BMIs less than 35, the relatively higher mechanical index resulted in no failures in shear wave elastography measurement, while the relatively lower mechanical index resulted in failures. Similarly, for BMIs over 35, the relatively higher mechanical index resulted in fewer failures in shear wave elastography measurement than the relatively lower mechanical index.

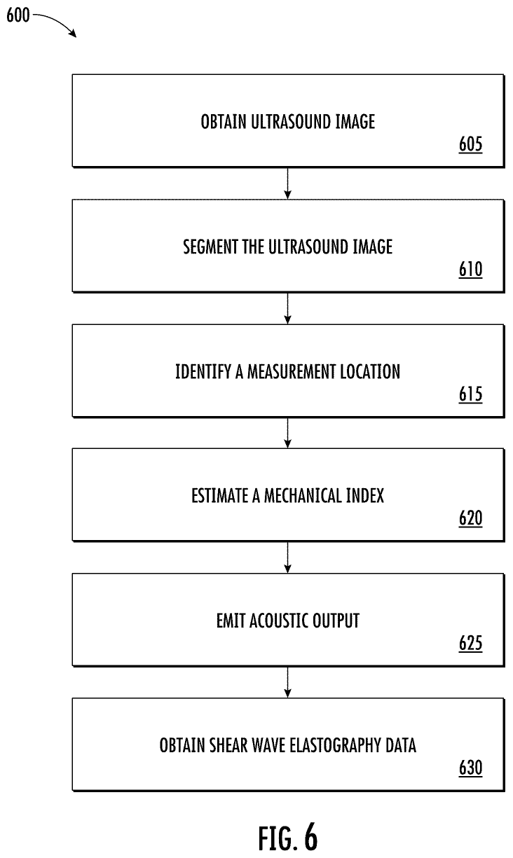

Referring to FIG. 6, a flow chart is shown illustrating a method 600 for performing a shear wave elastography process. In at least one embodiment, the ultrasound imaging system referred to by method 600 is the ultrasound imaging system 100 described above with reference to FIGS. 1-2, and method 600 may be implemented by the ultrasound imaging system 100. In some embodiments, method 600 may be implemented as executable instructions in a memory of the ultrasound imaging system 100, such as the memory 118 of FIG. 4.

As shown in FIG. 6, the method 600 may begin with obtaining an ultrasound image at step 605. More specifically, the ultrasound image obtained at step 605 may be a conventional B-mode image of a patient's liver. The ultrasound image obtained at step 605 may be the ultrasound image 300, as described above. In some embodiments, the ultrasound image is obtained by the probe 106, and may thereafter be processed by the image processing circuit 120.

After obtaining the ultrasound image at step 605, the ultrasound image may be segmented at step 610. That is, the segmentation circuit 122 may be configured to segment a liver (e.g., liver 305), a fat layer (e.g., fat layer 310), a SCD (e.g., SCD 315), large blood vessels, and so on, using a semi-supervised learning framework. In some instances, the segmentation circuit 122 may be configured to provide feedback at step 610 if an identified anatomical region (e.g., the liver 305) contains a vessel.

Based on the segmented ultrasound image, a measurement location is identified at step 615. Identifying the measurement location at step 615 includes estimating an optimal placement of the measurement location with the field of view depicted by the ultrasound image obtained at step 605. In this way, estimating the optimal placement of the measurement location allows for an estimation of the anatomical region that is most likely to yield a measurement of sufficient quality.

At step 620, method 600 includes estimating a mechanical index of an acoustic output. That is, step 620 includes estimating an optimal mechanical index for the measurement location identified at step 615. In some embodiments, the mechanical index is estimated at step 620 by the acoustic output circuit 124. As described in greater detail below with reference to FIG. 8, the mechanical index may be estimated at step 620 based on the SCD (e.g., SCD 315) from ultrasound image segmented at step 610. Additionally or alternatively, as described in greater detail below with reference to FIG. 9, the mechanical index may be estimated at step 620 based on a comparison of a patient's BMI to a threshold index. In some instances, estimating the mechanical index of the acoustic output at step 620 includes determining at least one of an amplitude or a shape of the acoustic output emitted during a shear wave elastography process.

At step 625, an acoustic output may be emitted at the measurement location identified at step 615 according to the mechanical index determined at step 620. The acoustic output refers to the amplitude of the push pulse from the transducer (e.g., the probe 106) that generates the shear waves that then propagate through the patient's tissue (e.g., the liver). In some embodiments, the acoustic output is emitted at step 625 by the acoustic output circuit 124.

In response to emitting the acoustic output at step 625, method 600 continues by obtaining shear wave elastography data at step 630. For instance, the shear elastography data obtained at step 630 may include the first shear wave elastography data 400a or the second shear wave elastography data 400b, as shown in FIG. 4.

Furthermore, method 600 may include repeating emission of the acoustic output according to the mechanical index based on determining that the mechanical index provides a sufficient force of the acoustic output based on the shear wave elastography data obtained at step 630. It should be appreciated that “sufficient force of the acoustic output,” as utilized herein, refers to a force of the acoustic output that is sufficient to produce a valid shear wave elastography measurement. Similarly, it should be appreciated that a “sufficient acoustic output,” as utilized herein, refers to an acoustic output that is sufficient to produce an acceptable image quality according to an imaging modality being used. Additionally or alternatively, method 600 may include determining, based on the shear wave elastography data obtained at step 630, that the mechanical index does not provide the sufficient force of the acoustic output. In such instances, method 600 may include increasing the mechanical index by an incremental amount and iteratively emitting the acoustic output according to the incrementally increased mechanical index until determining that the mechanical index provides the sufficient force of the acoustic output based on the shear wave elastography data.

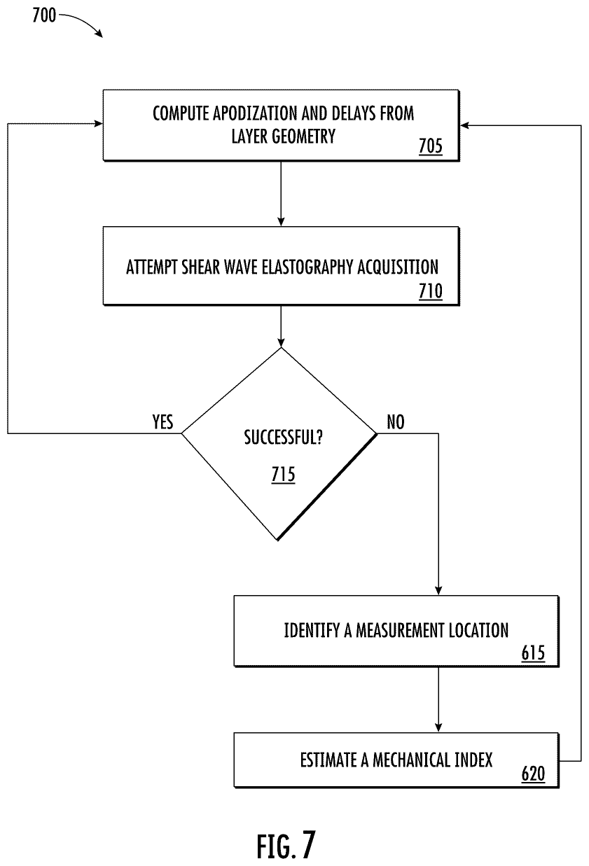

Referring to FIG. 7, a flow chart is shown illustrating a method 700 for iteratively performing the shear wave elastography process (e.g., method 600) of FIG. 6. In at least one embodiment, the ultrasound imaging system referred to by method 700 is the ultrasound imaging system 100 described above with reference to FIGS. 1-2, and method 700 may be implemented by the ultrasound imaging system 100. In some embodiments, method 700 may be implemented as executable instructions in a memory of the ultrasound imaging system 100, such as the memory 118 of FIG. 4.

As shown in FIG. 7, method 700 may begin with computing apodization and delays from layer geometry (e.g., fat layer 310) at step 705. Computing apodization and delays from layer geometry at step 705 refers to using the information from the segmented ultrasound image (e.g., segmented ultrasound image 300, the ultrasound image segmented at step 610 of method 600, etc.) to adjust the strength of each element (e.g., from the transducer elements 108, as described above) that contributes to the push beam (e.g., the acoustic output). That is, because the path of some energy from the elements to the intended focus may pass through varying amounts of fat (e.g., as shown by the asymmetry of the fat layer 310 in FIG. 3B), step 705 provides for an adjustment to the element amplitude and phase to compensate for the asymmetry of the fat layer 310. Such adjustments may also be applied to the received data for reducing aberration in the images used to track the shear waves.

Then, shear wave elastography acquisition is attempted at step 710. Method 700 continues by determining whether the shear wave elastography acquisition attempted at step 710 was successful at step 715. Determining whether the shear wave elastography acquisition attempted at step 710 was successful may include determining whether the mechanical index (e.g., estimated at step 620 of method 600) provides a sufficient force of the acoustic output based on shear wave elastography data (e.g., obtained at step 630 of method 600). For instance, as shown in FIG. 7, if step 715 includes determining that the shear wave elastography acquisition attempted at step 710 was successful, method 700 returns to computing apodization and delays from layer geometry at step 705. That is, in such instances, method 700 includes repeating emission of the acoustic output according to the mechanical index based on determining that the mechanical index provides the sufficient force of the acoustic output based on the shear wave elastography data.

If step 715 includes determining that the shear wave elastography acquisition attempted at step 710 was unsuccessful, method 700 returns to step 615 method 600 (e.g., identifying a measurement location). That is, in such instances, step 715 includes determining, based on the shear wave elastography data, that the mechanical index does not provide the sufficient force of the acoustic output. Then, as described above with reference to FIG. 6, a mechanical index is estimated at step 620 based on the measurement location identified at step 615. More specifically, the mechanical index may be increased during each iteration of step 615 by an incremental amount (e.g., ΔP, as described below). After estimating the mechanical index, method 700 returns to computing apodization and delays from layer geometry at step 705. In other words, such implementations of method 700 may include iteratively emitting the acoustic output according to the incrementally increased mechanical index until determining that the mechanical index provides the sufficient force of the acoustic output based on the shear wave elastography data.

Referring to FIG. 8, a flow chart is shown illustrating a method 800 for determining a mechanical index based on a SCD. In at least one embodiment, the ultrasound imaging system referred to by method 800 is the ultrasound imaging system 100 described above with reference to FIGS. 1-2, and method 800 may be implemented by the ultrasound imaging system 100. In some embodiments, method 800 may be implemented as executable instructions in a memory of the ultrasound imaging system 100, such as the memory 118 of FIG. 4.

As shown in FIG. 8, method 800 may include measuring a SCD at step 805. In some instances, the SCD measured at step 805 refers to the SCD 315 shown in FIG. 3B. That is, the SCD may be measured at step 805 by the segmentation circuit 122 based on an ultrasound image depicting a liver, such as the ultrasound image 300. Furthermore, measuring the SCD may be measured during segmentation of an ultrasound image (e.g., step 610 of method 600).

After measuring the SCD at step 805, the SCD is compared to a threshold distance at a measurement location at step 810. Based on the comparison of the SCD to the threshold distance at step 810, method 800 continues with determining whether the SCD is greater than the threshold distance at step 815.

If the SCD is greater than the threshold distance, method 800 includes applying an increased mechanical index at the measurement location at step 820a. For instance, if the SCD is greater than a threshold distance d(e.g., 1 cm, etc.), the mechanical index applied at step 820 a may be a default mechanical index MI+ΔP, where AP is an incrementally increased amount based on the SCD.

If the SCD is less than the threshold distance, method 800 includes applying a default mechanical index at the measurement location at step 820b. For instance, if the SCD is less than a maximum value (e.g., the threshold distance d), the mechanical index applied at step 820b may be the default mechanical index MI. If the SCD is equal to the threshold distance d, method 800 may include applying the default mechanical index MI at the measurement location at step 820b.

Referring to FIG. 9, a flow chart is shown illustrating a method 900 for determining a mechanical index based on a BMI. In at least one embodiment, the ultrasound imaging system referred to by method 900 is the ultrasound imaging system 100 described above with reference to FIGS. 1-2, and method 900 may be implemented by the ultrasound imaging system 100. In some embodiments, method 900 may be implemented as executable instructions in a memory of the ultrasound imaging system 100, such as the memory 118 of FIG. 4.

As shown in FIG. 9, method 900 begins with determining a patient BMI at step 905. In some instances, the patient BMI may be received at step 905 from patient medical records, as an input via the user interface 130, and so on. Furthermore, the patient BMI may be input directly or may be calculated using a height and a weight of the patient.

After the BMI is determined, the BMI is compared to a threshold index at step 910. Based on the comparison of the BMI to the threshold index at step 910, method 900 continues with determining whether the BMI is greater than the threshold index at step 915.

If the BMI is greater than the threshold index, method 900 includes applying an increased mechanical index at the measurement location at step 920a. For instance, if the BMI is greater than a threshold index B (e.g., 29), the mechanical index applied at step 920a may be a default mechanical index MI+ΔP, where ΔP is an incrementally increased amount based on the BMI. Furthermore, after a failed attempt of shear wave elastography acquisition (e.g., step 710 of method 700), the mechanical index may be increased (e.g., at step 620) by the incremental amount AP. If the BMI is equal to the threshold index B, method 900 may include applying the default mechanical index MI at the measurement location at step 920a.

If the BMI is less than the threshold index, method 900 includes applying a default mechanical index at the measurement location at step 920b. For instance, if the BMI is less than a minimum value B, the mechanical index applied at step 920b may be the default mechanical index MI.

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As utilized herein, terms of degree such as “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to any precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that terms such as “exemplary,” “example,” and similar terms, as used herein to describe various embodiments, are intended to indicate that such embodiments are possible examples, representations, or illustrations of possible embodiments, and such terms are not intended to connote that such embodiments are necessarily extraordinary or superlative examples.

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any element on its own or any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the drawings. It should be noted that the orientation of various elements may differ according to other exemplary embodiments, and that such variations are intended to be encompassed by the present disclosure.

As used herein, terms such as “engine” or “circuit” may include hardware and machine-readable media storing instructions thereon for configuring the hardware to execute the functions described herein. The engine or circuit may be embodied as one or more circuitry components including, but not limited to, processing circuitry, network interfaces, peripheral devices, input devices, output devices, sensors, etc. In some embodiments, the engine or circuit may take the form of one or more analog circuits, electronic circuits (e.g., integrated circuits (IC), discrete circuits, system on a chip (SOCs) circuits, etc.), telecommunication circuits, hybrid circuits, and any other type of circuit. In this regard, the engine or circuit may include any type of component for accomplishing or facilitating achievement of the operations described herein. For example, an engine or circuit as described herein may include one or more transistors, logic gates (e.g., NAND, AND, NOR, OR, XOR, NOT, XNOR, etc.), resistors, multiplexers, registers, capacitors, inductors, diodes, wiring, and so on).

An engine or circuit may be embodied as one or more processing circuits comprising one or more processors communicatively coupled to one or more memory or memory devices. In this regard, the one or more processors may execute instructions stored in the memory or may execute instructions otherwise accessible to the one or more processors. The one or more processors may be constructed in a manner sufficient to perform at least the operations described herein. In some embodiments, the one or more processors may be shared by multiple engines or circuits (e.g., engine A and engine B, or circuit A and circuit B, may comprise or otherwise share the same processor which, in some example embodiments, may execute instructions stored, or otherwise accessed, via different areas of memory).

Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be provided as one or more suitable processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud based processor). Alternatively or additionally, the one or more processors may be internal and/or local to the apparatus. In this regard, a given engine or circuit or components thereof may be disposed locally (e.g., as part of a local server, a local computing system, etc.) or remotely (e.g., as part of a remote server such as a cloud based server). To that end, engines or circuits as described herein may include components that are distributed across one or more locations.

An example system for providing the overall system or portions of the embodiments described herein might include one or more computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some embodiments, the non-volatile media may take the form of ROM, flash memory (e.g., flash memory such as NAND, 3D NAND, NOR, 3D NOR, etc.), EEPROM, MRAM, magnetic storage, hard discs, optical discs, etc. In other embodiments, the volatile storage media may take the form of RAM, TRAM, ZRAM, etc. Combinations of the above are also included within the scope of machine-readable media. In this regard, machine-executable instructions comprise, for example, instructions and data which cause a general purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components, etc.), in accordance with the example embodiments described herein.

Although the drawings may show and the description may describe a specific order and composition of method steps, the order of such steps may differ from what is depicted and described. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions, and arrangement of the embodiments without departing from the scope of the present disclosure as expressed in the appended claims.

1. A system comprising:
a processing circuit comprising a processor coupled to a memory device storing instructions thereon that, when executed, cause the processing circuit to perform operations comprising:
obtaining an ultrasound image depicting an organ of a patient;
segmenting the ultrasound image, wherein segmenting the ultrasound image comprises extracting the organ depicted by the ultrasound image;
determining, using the segmented ultrasound image, a skin-to-capsule distance;
identifying, using the segmented ultrasound image, a measurement location;
estimating a mechanical index of an acoustic output based on a comparison of the skin-to-capsule distance at the measurement location to a threshold distance;
emitting the acoustic output at the measurement location according to the mechanical index;
obtaining data in response to emitting the acoustic output; [[and]]
determining, based on the data, that the mechanical index does not provide a sufficient acoustic output;
increasing the mechanical index by an incremental amount; and
iteratively emitting the acoustic output according to the incrementally increased mechanical index until determining that the mechanical index provides the sufficient acoustic output based on the data.

2. The system of claim 1, wherein estimating the mechanical index based on the comparison of the skin-to-capsule distance to the threshold distance comprises at least one of:
estimating a default mechanical index based on determining that the skin-to-capsule distance is equal to or less than the threshold distance; or
estimating the mechanical index to be greater than the default mechanical index based on determining that the skin-to-capsule distance is greater than the threshold distance.

3. (canceled)

4. The system of claim 1, wherein the operations further comprise:
receiving a body mass index of the patient; and
determining the incremental amount based on a comparison of the body mass index to a threshold index.

5. The system of claim 1, wherein the organ is a liver.

6. The system of claim 5, wherein the data is shear wave elastography data.

7. The system of claim 1, wherein the ultrasound image is segmented using a semi-supervised learning framework.

8. The system of claim 1, wherein the ultrasound image is a B-mode image.

9. The system of claim 1, wherein estimating the mechanical index of the acoustic output comprises determining at least one of an amplitude or a shape of the acoustic output.

10. The system of claim 1, wherein the operations further comprise:
receiving a body mass index of the patient; and
estimating the mechanical index of the acoustic output based on a comparison of the body mass index to a threshold index.

11. A system comprising:
an ultrasound probe;
a transducer configured to obtain ultrasound image data of a patient, and
a processing circuit comprising a processor coupled to a memory device storing instructions thereon that, when executed, cause the processing circuit to perform operations comprising:
obtaining an ultrasound image depicting a liver of the patient;
receiving data regarding a body mass index of the patient;
segmenting the ultrasound image, wherein segmenting the ultrasound image comprises extracting the liver depicted by the ultrasound image;
determining, using the segmented ultrasound image, a skin-to-capsule distance;
identifying, using the segmented ultrasound image, a measurement location;
estimating a mechanical index of an acoustic output based on at least one of a comparison of the skin-to-capsule distance at the measurement location to a threshold distance or a comparison of the body mass index of the patient to a threshold index;
emitting the acoustic output at the measurement location according to the mechanical index;
obtaining shear wave elastography data in response to emitting the acoustic output;
determining, based on the shear wave elastography data, that the mechanical index does not provide a sufficient force of the acoustic output;
increasing the mechanical index by an incremental amount; and
iteratively emitting the acoustic output according to the incrementally increased mechanical index until determining that the mechanical index provides the sufficient force of the acoustic output based on the shear wave elastography data.

12. The system of claim 11, wherein estimating the mechanical index based on the comparison of the skin-to-capsule distance to the threshold distance comprises at least one of:
estimating a default mechanical index based on determining that the skin-to-capsule distance is equal to or less than the threshold distance; or
estimating the mechanical index to be greater than the default mechanical index based on determining that the skin-to-capsule distance is greater than the threshold distance.

13. The system of claim 11, wherein estimating the mechanical index based on the comparison of the body mass index of the patient to the threshold index comprises at least one of:
estimating a default mechanical index based on determining that the body mass index of the patient is equal to or less than the threshold index; or
estimating the mechanical index to be greater than the default mechanical index based on determining that the body mass index of the patient is greater than the threshold index.

14. (canceled)

15. The system of claim 11, wherein the incremental amount is determined based on the comparison of the body mass index to the threshold index.

16. A method comprising:
obtaining, by a processing circuit, an ultrasound image depicting a liver of a patient;
segmenting, by the processing circuit, the ultrasound image, wherein segmenting the ultrasound image comprises extracting the liver depicted by the ultrasound image;
determining, by the processing circuit using the segmented ultrasound image, a skin-to-capsule distance;
identifying, by the processing circuit using the segmented ultrasound image, a measurement location;
estimating, by the processing circuit, a mechanical index of an acoustic output based on a comparison of the skin-to-capsule distance at the measurement location to a threshold distance;
emitting, by the processing circuit, the acoustic output at the measurement location according to the mechanical index;
obtaining, by the processing circuit, shear wave elastography data in response to emitting the acoustic output;
determining, by the processing circuit and based on the shear wave elastography data, that the mechanical index does not provide a sufficient force of the acoustic output;
increasing, by the processing circuit, the mechanical index by an incremental amount; and
iteratively emitting, by the processing circuit, the acoustic output according to the incrementally increased mechanical index until determining that the mechanical index provides the sufficient force of the acoustic output based on the shear wave elastography data.

17. The method of claim 16, wherein the method further comprises:
receiving, by the processing circuit, a body mass index of the patient; and
estimating, by the processing circuit, the mechanical index of the acoustic output based on a comparison of the body mass index to a threshold index.

18. (canceled)

19. The method of claim 16, wherein the method further comprises:
receiving, by the processing circuit, a body mass index of the patient; and
determining, by the processing circuit, the incremental amount based on a comparison of the body mass index to a threshold index.

20. The method of claim 16, wherein estimating the mechanical index based on the comparison of the skin-to-capsule distance to the threshold distance comprises at least one of:
estimating a default mechanical index based on determining that the skin-to-capsule distance is equal to or less than the threshold distance; or
estimating the mechanical index to be greater than the default mechanical index based on determining that the skin-to-capsule distance is greater than the threshold distance.
